(19) United States
(12) Patent Application Publication
(10) Pub. No.: US 2008/0249006 A1
(43) Pub. Date: Oct. 9, 2008

(54) Pharmaceutical Preparation for the Treatment of Shock

(75) Inventors: Peter Petzelbauer, Wien (AT); Kai Zacharowski, Dusseldorf (DE)

Correspondence Address: WORKMAN NYDEGGER, 60 EAST SOUTH TEMPLE, 1000 EAGLE GATE TOWER, SALT LAKE CITY, UT, 84111, US

(73) Assignee: FIBREX MEDICAL RESEARCH & DEVELOPMENT GESMBH, Wien (AT)

(21) Appl. No.: 10/596,103

(22) PCT Filed: Jun. 24, 2005
(86) PCT No.: PCT/AT2005/000228

§ 371 Date: May 30, 2006

(30) Foreign Application Priority Data
Jun. 25, 2004 (AT) A 1087/2004
Jan. 13, 2005 (AT) A 40/2005

(51) Int. Cl. A61K 38/16; A61K 38/06; A61P 25/00; A61K 38/08

(52) U.S. Cl. 514/12; 514/18; 514/17

(57) ABSTRACT

The invention relates to the use of a peptide of general Formula I

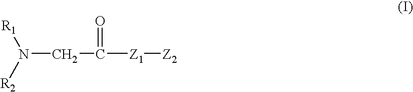

wherein
R₁ and R₂, being equal or different, denote hydrogen, a saturated or unsaturated hydrocarbon moiety comprising from 1 to 10, in particular from 1 to 3, carbon atoms,
Z₁ denotes a histidine or proline moiety,
Z₂ denotes an arginine moiety, a peptide moiety or a protein moiety comprising an initial arginine moiety, in particular comprising from 2 to 30 amino acids,
which peptide has the biological property of matching the inducible VE-cadherin binding motif on the Bβ-chain (i.e. Bβ₁₅₋₄₂) of human fibrin, for the preparation of a pharmaceutical preparation for the treatment of shock.

### BACKGROUND OF THE INVENTION

The present invention is directed to a pharmaceutical preparation for the treatment of shock.

Shock is an acute complication of many different pathological conditions characterized by the inability of the cardiovascular system to maintain an adequate perfusion pressure. Infectious agents can directly or indirectly cause a failure of the cardiovascular system. Bacteria, bacterial toxins, virus and last but not least an inadequate cellular or humoral host response involving inflammation and coagulation can lead to a loss of vascular tone, loss of vascular barrier function, loss of myocardial contractility and loss of organ function, which alone or in combination leads to shock and finally to the death of the patient. Treatment of bacterial infection relies on antibiotic treatment, which kills the bacteria but this does not treat toxinemia and does not correct for the inadequate cellular or humoral response. In Gram-negative bacteria lipopolysaccharide (LPS or endotoxin) is responsible for the initiation of Gram-negative shock. Gram-positive bacteria can cause multiple organ failure and septic shock without endotoxemia but the cell wall of Gram-positive bacteria also contains toxins like lipoteichoic acid (LTA) and peptidoglycan (PepG). LTA and PepG act in synergy to release cytokines such as tumor necrosis factor (TNF) α and interferon (IFN) γ, to induce iNOS and finally cause shock and organ failure.

Endotoxemia, sepsis and septic shock are associated with the generation of extensive amounts of nitric oxide (NO). The excessive vasodilatation and vascular hyporeactivity to pressor agents associated with circulatory shock can be reversed with inhibitors of the inducible isoform of NO synthase (iNOS) (Southan and Szabo, Biochem Pharmacol. 1996; 51:383-94, Thiemermann Gen Pharmacol 1997; 29:159-66), but iNOS inhibitors do not reduce the organ injury caused by toxins (Wray et al. Shock 1998; 9:329-335).

Treatment of shock caused by viral infections is even a greater challenge, since anti-viral drugs are not available for most infections. Treatments aiming to eliminate the infectious agent alone are not sufficient in patients with shock due to an infectious agent, because secondary events initiated by the infectious agent involving an inflammatory reaction and alterations in the coagulation system may have become independent and lead to the death of the patient irrespective of the question whether the causative infectious agent has been neutralized or not. A specific treatment is not available, thus current procedures aim to relieve symptoms, which includes mechanical ventilation, fluid replacement, the use of cardio active drugs, strict control of oxygen saturation, hemoglobin, glucose and renal function. The control of the inflammation reaction only, e.g. with high dosage steroids or the inhibition of coagulation with antithrombin, does not produce improvement of survival. The only molecule which so far has been proven to be of notable effectiveness in reducing mortality is activated protein C′, which interacts with coagulation/fibrinolysis and the inflammation processes.

Shock during the course of an infection is mostly associated with overt or non-overt changes in plasma fibrinogen accompanied by fibrin formation and by a raise of fibrin fragments. This activation of clotting as well as fibrinolytic pathways may result in overt or non-overt disseminated intravascular coagulation (DIC) resulting in vessel occlusion and end-organ damage, and in consumption of coagulation factors resulting in bleeding. Sepsis is the commonest cause of DIC. Importantly, fibrinogen, fibrin and fibrin fragments play not only a role in blood coagulation, but have several binding sites for cellular and matrix proteins, which allow them to interact with white blood cells, platelets, endothelial cells and matrix structures. This leads to cell activation, cell migration, cytokine release and ultimately to an inflammatory reaction. The role of fibrinogen or fibrin in inflammation is amply documented (reviewed by Altieri Thromb Haemost 82:781-786; Herrick et al. Int J Biochem Cell Biol 31:741-46). The D-region of the molecule contains many binding sites for matrix molecules, endothelial cells, platelets and inflammatory cells. The E-region of fibrin binds to CD11c (Loike et al. Proc Natl Acad Sci USA 88:1044-48).

We have recently described a novel role for the Bbeta₁₅₋₄₂ sequence of fibrin in inflammation (WO 02/48180). This sequence is also located within the E-region of fibrin and is only active when fibrinopeptide is cleaved. Fibrin fragments containing this sequence at their free N-terminus of the beta chain bind to endothelium and cause inflammation, and a peptide matching the amino acids 15-42 of the Bbeta chain of fibrin blocks binding of fibrin fragments to endothelial surfaces and blocks inflammation in vitro (WO 02/48180). In vivo, this peptide prevents myocardial inflammation and reduces myocardial infarct sizes in situations of ischemia/reperfusion (WO 02/48180).

Fibrin fragments occur in any situation of impaired fibrin formation and impaired fibrinolysis. Specifically in situations of shock due to an infectious agent, this altered fibrin formation and fibrinolysis is a major problem. For many diseases a direct correlation between the outcome and the impairment of fibrin formation/fibrinolysis has been documented. E.g. Dengue (van Gorp et al. J Med Virol 2002, 67:549-54, Mairuhu et al. Lancet Inf Dis 2003; 3:33-41). Adult respiratory distress syndrome (ARDS) is a form of acute lung injury that is characterized by florid extravascular fibrin deposition (Idell Am J Respir Med. 2002; 1:383-91). Thrombosis in the pulmonary vasculature and disseminated intravascular coagulation have also been observed in association with ARDS.

The reasons for the persistence/global emergence of Dengue fever (DF) and hemorragic Dengue fever (DHF) as a major public health problem are complex, vector control measures were not successful to eliminate DF/DHF. Currently the main focus of public sector funding for dengue research (estimated to be US$ 15 million in 2001) is on molecular epidemiology, immune pathophysiology, second generation vaccine discovery research, and new or improved approaches to vector control.

Several candidate vaccines are in clinical trial in USA and Thailand, but still no drug to treat infected patients is on the market, even worse, currently no commercial chemo-therapy R&D activities appear to be under way. The world health organization has published strategic directions to fight DF/DHF which—as high priority aims—include the development of antivirals directed at protease or other poorly studied enzymes; the development of anti-mediators directed at causes of increased vascular permeability or altered haemostasis.

### SUMMARY OF THE INVENTION

The invention relates to the use of a peptide of general Formula I

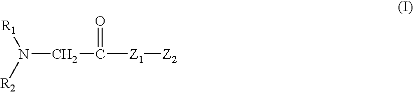

wherein
R₁ and R₂, being equal or different, denote hydrogen, a saturated or unsaturated hydrocarbon moiety comprising from 1 to 10, in particular from 1 to 3, carbon atoms,
Z₁ denotes a histidine or proline moiety,
Z₂ denotes an arginine moiety, a peptide moiety or a protein moiety comprising an initial arginine moiety, in particular comprising from 2 to 30 amino acids,
which peptide has the biological property of matching the inducible VE-cadherin binding motif on the Bβ-chain (i.e. Bβ₁₅₋₄₂) of human fibrin, for the preparation of a pharmaceutical preparation for the treatment of shock.

A peptide of general Formula II

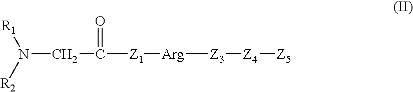

is preferably used, wherein
Z₁ denotes a histidine or proline moiety,
Arg denotes an arginine moiety,
Z₃ denotes a proline or valine moiety,
Z₄ denotes a leucine or valine moiety,
Z₅ denotes a peptide moiety or a protein moiety in particular comprising from 2 to 30 amino acids or an alcohol moiety comprising from 1 to 10, in particular from 1 to 3, carbon atoms or an organic or inorganic base moiety.

Furthermore, a peptide is preferably used in which Z₅ is a peptide moiety derived from the Aalpha-chain or the Bbeta-chain of the fibrin.

Moreover, a peptide is preferably used in which

Z₅ is a peptide moiety comprising the amino acid sequence

Asp Lys Lys Arg Glu Glu Ala Pro Ser Leu Arg Pro
Ala Pro Pro Ile Ser Gly Gly Gly Tyr Arg

Z₁ is a histidine moiety,
Arg is an arginine moiety,
Z₃ is a proline moiety, and
Z₄ is a leucine moiety.

Furthermore, a peptide is preferably used in which

Z₅ is a peptide moiety comprising the amino acid sequence

Glu Arg His Gln Ser Ala Cys Lys Asp Ser Asp Trp
Pro Phe Cys Ser Asp Glu Asp Trp Asn Tyr Lys

Z₁ is a proline moiety,
Arg is an arginine moiety,
Z₃ is a valine moiety, and
Z₄ is a valine moiety.

Furthermore, the invention relates to the use of a peptide which exhibits the N-terminal sequence

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Gly-Tyr-Arg

and which has the biological property of matching the inducible VE-cadherin binding motif on the Bβ-chain (i.e. Bβ₁₅₋₄₂) of human fibrin for the preparation of a pharmaceutical preparation for the treatment of shock.

A further preferred embodiment of the use according to the invention is characterized in that the peptide is

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Gly-Tyr-Arg.

It has been shown that in particular shock conditions can be treated with the above-mentioned peptides, wherein shock is associated with one or more from the group comprising bacterial toxins, disseminated intravascular coagulopathy, necrotizing fasciitis, haemorrhagic shock following viral infection, in particular caused by filovirus, arenaviridae, bunyaviridae, flavivirus, dengue, acute hemorrhagic respiratory failure caused by infectious agents or autoimmune diseases, organ failure after organ injury, in particular myocardial infarction, vascular surgery, clamping of organs, haemorrhagic shock, lung infarction, liver infarction, gut infarction, surgical procedures and stroke, and organ dysfunction of grafted organs.

### DETAILED DESCRIPTION OF THE INVENTION

### Peptides and Proteins

Peptides were produced by solid-phase peptide synthesis and purified with reversed-phase HPLC using nucleosil 100-10C18 columns (PiChem, Graz, Austria). It should be noted that the beta 15-42 region is 100% similar among species when allowing for conservative amino acid substitutions. The N-terminal disulfide knot of fibrinogen (NDSK) composed of amino acids Aα1-51, Bβ1-118 and γ1-78 was prepared as previously described (WO 02/48180). The N-terminal disulfide knot of fibrin (NDSK-II, which lacks fibrinopeptides A and B) composed of amino acids Aα17-51, Bβ15-118 and γ1-78 was prepared by treating NDSK with thrombin (20 U/1 mg of NDSK) for 3 h at 37° C. Residual thrombin was neutralized with 10 mM disopropyl fluorophosphate (Fluka, Milwaukee, Wis.) for 2 h at 37° C. All products were then dialyzed into phosphate buffered saline (PBS).

### Elisa

Peptide Bβ₁₅₋₄₂ binds to VE-cadherin

The interaction of the Bbeta chain (Bbeta₁₅₋₄₂) of fibrin with endothelial cells causes morphologic changes (Bunce et al. J Clin Invest 89:842-50; Bach et al. Exp Cell Res 238:324-34; Chalupowicz et al. J Cell Biol 130:207-15; Hamaguchi et al. Blood 81:2348-56; Francis et al. Blood cells 19:291-306), proliferation (Spom et al. Blood 86:1802-10), the release of von Willebrand factor (Ribes et al. J Clin Invest 79:117-23, Ribes et al. J Clin Invest 84: 435-42; Erban and Wagner, J Biol Chem 267, 2451-58) and possibly IL-8 (Qi et al. Blood 90:3593-3602) and membrane expression of CD54 (Harley et al. Art Thromb Vasc Biol 20:652-658). VE-cadherin has been identified as a binding ligand of the sequence Bbeta₁₅₋₄₂ and ELISAs have been developed to demonstrate this interaction of endothelial cells and/or VE-cadherin with fibrin or fibrin fragments. Martinez et al. have used anti-pan cadherin antibodies to capture cadherins from endothelial cells followed by incubation with fibrin (Martinez et al. Ann NY Acad Sci 936:386-405), HUVEC monolayers (which express VE-cadherin) have been overlaid with radio-labeled fibrin fragments or peptide Bbeta₁₅₋₄₂ (Bach et al. J Biol Chem 273:30719-28; Harley et al. Art Thromb Vasc Biol 20:652-658), and recombinant VE-cadherin was used by Gorlatov and Medved (Biochemistry 41:4107-16). Others have used ELISA for detection of fibrin fragments within the blood, mostly by using antibodies to distinct sequences within the fibrinogen molecule including antibodies against the Bbeta₁₅₋₄₂ motif (reviewed in Fareed et al. Clin Chem 8:1845-53).

We have developed a modified ELISA working with the same principles described by others, but the purpose of the herein described ELISA is not to quantify fibrin degradation products, but to search for proteins, peptides or compounds which interfere with the binding of the Bbeta₁₅₋₄₂ sequence and the VE-cadherin. The principle is that the VE-cadherin, either as a truncated protein, as a full protein or coupled with other proteins which do not interfere with the Bbeta₁₅₋₄₂ binding site, is allowed to interact with the Bbeta₁₅₋₄₂ sequence of fibrin. Into this system one can introduce any other additional substance and measure if this substance inhibits VE-cadherin/Bbeta₁₅₋₄₂ binding.

In detail, 96 well protein immobilizer plates (Exiqon, Vedbaek, D K) were coated with recombinant human VE-cadherin FC fusion protein (8 nM/ml; R&D Systems, Minneapolis) in PBS and were left overnight at 4° C. Plates were then washed and incubated with peptide Bβ₁₅₋₄₂ (GHRPLDKKREEAPSLRPAPPPISGGGYR) tagged with a FLAG-sequence (DYKDDDDK) at the C-terminus of the peptide or with a FLAG-tagged random peptide (DRGAPAHRPPRGPISGRSTPEKEKLLPG) at a concentration of 0-80 μMol. After washing, bound FLAG-tagged peptide was detected by incubation with a peroxidase-labelled anti-FLAG antibody (Sigma, St. Louis, USA) and chromogenic substrate. Optical density was determined by an ELISA plate reader set at a wavelength of 450 nm. Data represent the mean of three independent experiments, each performed in triplicates. The table below shows that the peptide Bβ₁₅₋₄₂ bound to VE-cadherin in a concentration-dependent manner. In contrast, the random peptide demonstrated only insignificant binding.

Dose dependent binding of peptide Bbeta₁₅₋₄₂ to VE-cadherin

| | | μM | | | | | | | | | |
|---|---|---|---|---|---|---|---|---|---|---|---|
| | | 0 | 0.23 | 0.7 | 2.3 | 7 | 14 | 21 | 35 | 46 | 70 |
| 15-42 | mean | 0 | 0.01 | 0.02 | 0.08 | 0.33 | 0.92 | 1.3 | 1.5 | 1.93 | 2.1 |
| FLAG | SD | 0 | 0.01 | 0.01 | 0.03 | 0.17 | 0.19 | 0.2 | 0 | 0 | 0 |
| random | mean | 0 | 0.01 | 0 | 0.01 | 0.03 | 0.12 | 0.2 | | 0.35 | 0.5 |
| FLAG | SD | 0 | 0.01 | 0 | 0.01 | 0.02 | 0.04 | 0.1 | | 0 | 0 |

Peptide B 15-42 and fibrin fragments compete for binding to VE-cadherin.

In a next step, we analyzed whether this ELISA can be used to screen for other peptides/compounds to compete with the binding of the Bβ₁₅₋₄₂ sequence to VE-cadherin. As expected, peptide Bβ₁₅₋₄₂ completely inhibited binding of the flag-tagged peptide Bβ₁₅₋₄₂ and was used as the positive control and random peptides or solvent had no effect and were used as negative controls. Shorter peptides partially inhibited the binding of Bβ₁₅₋₄₂ to VE-cadherin. NDSK-II inhibited Bβ₁₅₋₄₂ binding in a concentration-dependent fashion. An equilibrium between Bβ₁₅₋₄₂ and NDSK-II (50% inhibition) was reached at a molar ratio of 24:1. NDSK had little or no effect.

VE-cadherin was coated to the plastic surface at a concentration of 8 nM. Then indicated peptides were added at concentrations of 200 μM, NDSK or NDSK-II were added at indicated concentrations. Detection of binding of the FLAG-tagged Bbeta₁₅₋₄₂ (12 μM) was performed as described above.

| | % inhibition of |
|---|---|
| | 15-42FLAG-binding |
| | to VE-cadherin |
| Blocking reagent | mean ± SD |
| peptide 15-42 (28mer) | 100 ± 10 |
| peptide random (4mer) | 3 ± 3 |
| peptide random (28mer) | 10 ± 3 |
| solvent | 0 + 0 |
| peptide 15-18 (4mer) 200 μM | 65 ± 12 |
| peptide 15-26 (12mer) 200 μM | 64 ± 10 |
| peptide 15-30 (16mer) 200 μM | 61 ± 13 |
| peptide 15-34 (20mer) 200 μM | 67 ± 17 |
| peptide 15-37 (24mer) 200 μM | 17 ± 19 |
| peptide 16-42 (27mer) 200 μM | 55 ± 13 |
| peptide 15-18 (4mer) 12 μM | 7 ± 2 |
| peptide 15-26 (12mer) 12 μM | 6 ± 1 |
| peptide 15-30 (16mer) 12 μM | 6 ± 3 |
| peptide 15-34 (20mer) 12 μM | 7 ± 1 |
| peptide 15-37 (24mer) 12 μM | 7 ± 2 |
| peptide 16-42 (27mer) 12 μM | 5 ± 2 |
| NDSK-II 0.06 μM | 1 + 0 |
| NDSK-II 0.12 μM | 39 + 18 |
| NDSK-II 0.20 μM | 42 + 14 |
| NDSK-II 0.60 μM | 52 + 16 |
| NDSK-II 1.2 μM | 63 + 13 |
| NDSK-II 2.4 μM | 79 + 9 |
| NDSK-II 4.0 μM | 82 + 12 |
| NDSK 0.06 μM | 0 + 0 |
| NDSK 0.12 μM | 2 + 1 |
| NDSK 0.20 μM | 1 + 1 |
| NDSK 0.60 μM | 7 + 6 |
| NDSK 1.2 μM | 15 + 13 |
| NDSK 2.4 μM | 16 + 9 |
| NDSK 4.0 μM | 20 + 10 |
| anti-VE-cadherin Ab (TEA1/31, 1 mg/ml) | 2 + 1 |

Effectiveness of peptide bbeta₁₅₋₄₂ for the treatment of dengue virus infected mice.

### Materials and Methods.

Virus. Dengue virus type 2 (DEN-2), strain P23085, was obtained from the State Collection of Viruses, Moscow, Russia in the form of infected ICR mouse brain lyophilized suspension. The obtained Dengue virus has undergone passages in the brain of the suckling ICR mice as described earlier (Atrasheuskaya et al. FEMS Immunology and Medical Microbiology. 35, 33-423). Ten % brain suspension served as a virus stock and was stored at −40° C. The virus titer was determined by the serial dilutions of brain suspension. Brain suspension was inoculated i.p. in groups of 10 mice (4-week old BALB/c) each and the mortality was recorded. The virus titer was calculated, and it was at 7.4 lg LD50/ml. All work with the infectious virus was performed in the maximum containment biosafety level-3 (BSL-3) laboratory of the SRC VB<<Vector>> (Russia). Animals.

Four-week old inbred male BALB/c mice (haplotype H-2d) were received from the vivarium of the State Research Center of Virology and Biotechnology <<Vector>>. Animals were placed in individual cages with food and water available ad libitum. Assays.

Blood was taken from mice under methoxyflurane anesthesia from the orbital sinus before infection and after the challenge with DEN-2. Three mice for each time point were used for harvesting blood.

Circulating platelets (PLT), red blood cells (RBC), white blood cells (WBC), hemoglobin (HGB) and hematocrit (HCT) were determined by using Cell-Dyn 900 Hematology analyzer (Sequoia-Turner corporation, USA, CA).

Part of the harvested blood was centrifuged for obtaining serum, which was stored at −80° C. till the end of experiment. Serum levels of cytokines were measured by using enzyme immunoassay kits produced by R&D Systems (Minneapolis, USA) according to the manufacture's instructions. Detection limits were as follows: TNF-α, less 5.1 pg/ml; interleukin (IL)-1β, 3.0 pg/ml; IL-6, 3.1 pg/ml; IFNγ-less 20 pg/ml.

Dengue virus in the animals' blood was identified by RT-PCR as described earlier (Harris et al. J. Clin. Microbiol. 36, 2634-2639). Total RNA from the blood was isolated using a kit from Quiagen (Germany). Primers were as following: upper 5′AATATGCTGAAACGCGAGAGAAACCG (position 136-161), lower 5′AAGGAACGCCACCAAGGCCATG (position 237-258), amplifying a 119 bp product. To quantify the virus load, DEN-2 was titrated onto Vero E6 cell cultures as described earlier (Harris et al. J. Clin. Microbiol. 36, 2634-2639). On day 0 and 22 after the challenge blood of the surviving mice was analyzed for anti-DEN-2 antibodies (IgG) by ELISA as described earlier (Ignatyev et al. J. Biotechnology. 44, 111-118). Design of experiment.

Inbred four-week old male BALB/c mice were divided into 6 main groups. Each group contained 50 mice. All animals were infected intraperitoneally (i.p.) with the mouse-adapted DEN-2 strain P23085 (as described above) with a dose of 1 LD₅₀ and examined daily for signs of morbidity. Mice from the first subgroups of all main groups (A1-F1) were used for the mortality control. Each subgroup contained 20 mice. Animals of the second subgroups (A2-F2) were used for obtaining serum samples. Each subgroup contained 30 mice.

Group description. n=50 in each group
Control group received only virus.

Treatment with peptide Bβ₁₅₋₄₂ was performed twice per day, 4800 μg/kg each by intraperitoneal injection from day 3-post infection to day 8-post infection.

Blood and serum samples were obtained at the selected time points: day 1, 3, 5, 7, 11, 22 after the challenge.
Statistical analysis was conducted using Student's t or Chi-square test. P values <0.05 were considered significant.

TABLE 1
Mortality and IgG titer. p < 0.05 between groups

| | Mortality | Survival | | |
|---|---|---|---|---|
| Group | (%) | (%) | mean time to death. | IgG titer |
| untreated | 40 | 60 | 6.800 ± 0.245 | 1:160 |
| Bβ₁₅₋₄₂ | 0 | 100 | All mice survived | 1:20 |

TABLE 2

| | controls | | | Bβ₁₅₋₄₂-treated | | |
|---|---|---|---|---|---|---|
| | day 3 | 5 | 7 | day 3 | 5 | 7 |
| hemoglobin g/ml | 14 | 10 | 10 | 15 | 10 | 15 |
| hematokrit % | 15 | 22 | 35 | 15 | 33 | 43 |
| TNF pg/ml | 33 | 71 | 65 | 32 | 65 | 45 |
| IL-6 pg/ml | 210 | 210 | 150 | 140 | 110 | 100 |
| IL-1 pg/ml | 32 | 55 | 59 | 32 | 29 | 28 |

viremia 1 g PFU/ml

| | controls | Bb₁₅₋₄₂ |
|---|---|---|
| day | 0 | 0 |
| 2 | 1.2 ± 0.1 | 1.2 ± 0.2 |
| 3^{b} | 2.4 ± 0.3 | 2.2 ± 0.2 |
| 4 | 4.4 ± 0.2 | 4.2 ± 0.2 |
| 5 | 6.0 ± 0.4 | 5.6 ± 0.4 |
| 6 | 6.2 ± 0.4 | 6.0 ± 0.4 |
| 7 | 6.3 ± 0.3 | 5.9 ± 0.4 |
| 28 | 0- | 0 |

Gram-negative shock

Male Wistar rats weighing 230-280 g were housed in the Tierversuchsanlage (University of Düisseldorf) and fed on a standard diet with water ad libitum. All procedures were carried out in accordance with the AAALAC guidelines and Guide for the Care and Use of Laboratory Animals (Department of Health and Human Services, National Institutes of Health, Publication No. 86-23). In addition, all experiments have been approved by an ethical and research board of the University of Düisseldorf and the county. As previously described (Zacharowski et al. Crit. Care Med 2000, Zacharowski et al. Crit. Care Med 2001; 29:1599-1608), rats were anaesthetized with sodium thiopentone (120 mg/kg i.p.) and anesthesia was maintained with supplementary doses of sodium thiopentone as required.

The trachea was cannulated to facilitate respiration and rectal temperature was maintained at 37° C. with a homoeothermic blanket. The right carotid artery was catheterized and connected to a pressure transducer for the measurement of phasic and mean arterial blood pressure (MAP) and heart rate (HR) which were displayed on a data acquisition system (MacLab 8e, ADI Instruments, Germany) installed on an IBM computer. The right jugular vein was cannulated for the administration of drugs. The bladder was also cannulated to facilitate urine flow and to prevent the possibility of the development of post-renal failure. All animals received a total fluid replacement of 1.0 ml/kg/h (0.9% sodium chloride, saline, as an i.v. infusion into the jugular vein) throughout the experiment. Upon completion of the surgical procedure, cardiovascular parameters were allowed to stabilize for 15 min and constantly recorded over 6 h. In this model of LPS-induced multiple organ failure, a period of 6 h is essential to achieve a significant rise in the serum levels of AST and ALT, while a significant rise in the serum levels of urea and creatinine can already be observed after 2 h.

Three groups were studied:
Rats were subjected to sham operation: (sham).
Rats were subjected to Gram-negative shock. Lipopolysaccharide from E. coli, serotype 0.127:B8 (6 mg/kg i.v.) was given over 5 min i.v., 1 h later, animals received saline (2.4 ml/kg): (LPS+saline).
Rats were subjected to Gram-negative shock. Lipopolysaccharide from E. coli, serotype 0.127:B8 (6 mg/kg i.v.) was given over 5 min i.v., animals received Bβ₁₅₋₄₂ (2.4 mg/kg): (LPS+Bβ₁₅₋₄₂).

Survival n = 20 in each group, p < 0.05

| sham | LPS plus saline | LPS plus Bβ₁₅₋₄₂ |
|---|---|---|
| 100% | 25% | 88% |

Six hours after the induction of Gram-negative shock, blood was collected from the catheter placed in the right carotid artery. The blood sample was centrifuged (1610×g for 3 min at room temperature) to separate plasma. The following marker enzymes were measured in the plasma as biochemical indicators of multiple organ injury/dysfunction:

Liver injury was assessed by measuring the rise in plasma levels of alanine aminotransferase (ALT, a specific marker for hepatic parenchymal injury) and aspartate aminotransferase (AST, a non-specific marker for hepatic injury).

Renal dysfunction was estimated by measuring the rises in plasma levels of urea (an indicator of impaired excretory function of the kidney and/or increased catabolism) and creatinine (an indicator of reduced glomerular filtration rate, and hence, renal dysfunction). Plasma levels of glucose and amylase were measured as indirect markers of pancreatic function and injury.

In addition, arterial pO₂ was measured as indirect marker of lung function/injury. Laboratory values

| | sham | control | Bβ₁₅₋₄₂ |
|---|---|---|---|
| ALT | 39.8 | 542.3 | 261.9 |
| SEM | 5.2 | 117.2 | 42.7 |
| AST | 194.1 | 908.8 | 529.0 |
| SEM | 30.8 | 140.9 | 75.7 |
| Creatinine | 0.5 | 0.9 | 0.6 |
| SEM | 0.0 | 0.1 | 0.1 |
| Urea | 49.1 | 123.2 | 107.8 |
| SEM | 5.8 | 4.6 | 5.9 |
| Glucose | 131.5 | 75.3 | 45.8 |
| SEM | 7.5 | 5.4 | 9.1 |
| Amylase | 1713.3 | 1837.4 | 1945.1 |
| SEM | 131.5 | 122.7 | 176.8 |
| pO2 | 90.0 | 67.0 | 98.7 |
| SEM | 1.8 | 3.7 | 8.2 |

mean arterial pressure

| | | | |
|---|---|---|---|
| | | | LPS + |
| time | sham | LPS | Bβ₁₅₋₄₂ |

| (h) | mean | SEM | mean | SEM | mean | SEM |
|---|---|---|---|---|---|---|
| 0 | 120.9 | 5.5 | 118.9 | 5.4 | 128.9 | 5.2 |
| 1 | 111.5 | 10.6 | 90.7 | 5.5 | 83.7 | 4.6 |
| 2 | 113.2 | 7.3 | 100.2 | 5.1 | 102.3 | 4 |
| 3 | 116.4 | 5.4 | 90 | 7.4 | 103.2 | 4 |
| 4 | 108.7 | 8.9 | 81.1 | 7.9 | 101.2 | 3 |
| 5 | 104.1 | 9.6 | 60.1 | 10.8 | 97.1 | 5.4 |
| 6 | 104.1 | 9.6 | 34.1 | 7.7 | 107.7 | 9.6 |

heart rate

| | | | |
|---|---|---|---|
| | | | LPS |
| time | sham | LPS | Bβ₁₅₋₄₂ |

| (h) | mean | SEM | mean | SEM | mean | SEM |
|---|---|---|---|---|---|---|
| 0 | 482.2 | 17 | 457 | 18.1 | 436.9 | 6.4 |
| 1 | 461.7 | 12.1 | 511.2 | 27.4 | 484.8 | 18.6 |
| 2 | 488.1 | 13.6 | 523.3 | 27.3 | 484.1 | 10.3 |
| 3 | 506.6 | 26.6 | 518.5 | 24.9 | 509.8 | 12 |
| 4 | 488.9 | 17.4 | 516.7 | 32.1 | 516.7 | 13.4 |
| 5 | 470.4 | 13.9 | 515.5 | 26.1 | 533.7 | 30.7 |
| 6 | 443.7 | 0.7 | 530.1 | 27.7 | 541.6 | 24.4 |

Organ (lung, liver, heart and kidney) biopsies of all groups studied were taken at the end of the experiment. The biopsies were fixed in buffered formaldehyde solution (4% in phosphate buffered saline) at room temperature and sent to Vienna. Standard H&E-stained sections revealed no differences. However, in sections stained for fibrin deposits using acidic fuchsin—orange G, we found significantly elevated numbers of fibrin thrombi in controls receiving LPS alone as compared with animals treated with LPS plus Bβ₁₅₋₄₂ (p<0.05). In sham treated animals no fibrin thrombi were present.

mean number of fibrin thrombi within vessels

| | LPS plus saline | LPS plus Bbeta₁₅₋₄₂ |
|---|---|---|
| heart | 28 + 13 | 5 + 2 |
| kidney | 17 + 8 | 2 + 9 |
| liver | 47 + 41 | 78 + 37 |
| lung | 1.7 + 1 | 1 + 0 |

1. The use of a peptide of general Formula I 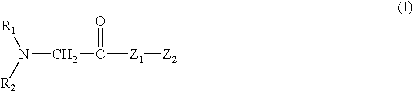
wherein
R₁ and R₂, being equal or different, denote hydrogen, a saturated or unsaturated hydrocarbon moiety comprising from 1 to 10, in particular from 1 to 3, carbon atoms,
Z₁ denotes a histidine or proline moiety,
Z₂ denotes an arginine moiety, a peptide moiety or a protein moiety comprising an initial arginine moiety, in particular comprising from 2 to 30 amino acids,
which peptide has the biological property of matching the inducible VE-cadherin binding motif on the Bβ-chain (i.e. Bβ₁₅₋₄₂) of human fibrin, for the preparation of a pharmaceutical preparation for the treatment of shock.

2. The use according to claim 1, characterized in that the peptide exhibits the general Formula II 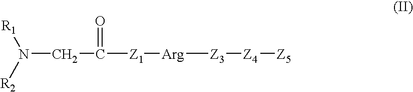
wherein
Z₁ denotes a histidine or proline moiety,
Arg denotes an arginine moiety,
Z₃ denotes a proline or valine moiety,
Z₄ denotes a leucine or valine moiety,
Z₅ denotes a peptide moiety or a protein moiety in particular comprising from 2 to 30 amino acids or an alcohol moiety comprising from 1 to 10, in particular from 1 to 3, carbon atoms or an organic or inorganic base moiety.

3. The use according to claim 2, characterized in that Z₅ is a peptide moiety derived from the Aalpha-chain of the fibrin.

4. The use according to claim 2, characterized in that Z₅ is a peptide moiety derived from the Bbeta-chain of the fibrin.

5. The use according to claim 2, characterized in that
Z₅ is a peptide moiety comprising the amino acid sequence
Asp Lys Lys Arg Glu Glu Ala Pro Ser Leu Arg Pro
Ala Pro Pro Ile Ser Gly Gly Gly Tyr Arg
Z₁ is a histidine moiety,
Arg is an arginine moiety,
Z₃ is a proline moiety, and
Z₄ is a leucine moiety.

6. The use according to claim 2, characterized in that
Z₅ is a peptide moiety comprising the amino acid sequence
Glu Arg His Gln Ser Ala Cys Lys Asp Ser Asp Trp
Pro Phe Cys Ser Asp Glu Asp Trp Asn Tyr Lys
Z₁ is a proline moiety,
Arg is an arginine moiety,
Z₃ is a valine moiety, and
Z₄ is a valine moiety.

7. The use of a peptide which exhibits the N-terminal sequence
Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Gly-Tyr-Arg
which peptide has the biological property of matching the inducible VE-cadherin binding motif on the Bβ-chain (i.e. Bβ₁₅₋₄₂) of human fibrin, for the preparation of a pharmaceutical preparation for the treatment of shock.

8. The use according to claim 7, characterized in that the peptide is
Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Gly-Tyr-Arg.

9. The use according to claim 1, wherein shock is associated with one or more from the group comprising bacterial toxins, disseminated intravascular coagulopathy, necrotizing fasciitis, haemorrhagic shock following viral infection, in particular caused by filovirus, arenaviridae, bunyaviridae, flavivirus, dengue, acute hemorrhagic respiratory failure caused by infectious agents or autoimmune diseases, organ failure after organ injury, in particular myocardial infarction, vascular surgery, clamping of organs, haemorrhagic shock, lung infarction, liver infarction, gut infarction, surgical procedures and stroke, and organ dysfunction of grafted organs.

10. The use according to claim 2, wherein shock is associated with one or more from the group comprising bacterial toxins, disseminated intravascular coagulopathy, necrotizing fasciitis, haemorrhagic shock following viral infection, in particular caused by filovirus, arenaviridae, bunyaviridae, flavivirus, dengue, acute hemorrhagic respiratory failure caused by infectious agents or autoimmune diseases, organ failure after organ injury, in particular myocardial infarction, vascular surgery, clamping of organs, haemorrhagic shock, lung infarction, liver infarction, gut infarction, surgical procedures and stroke, and organ dysfunction of grafted organs.

11. The use according to claim 3, wherein shock is associated with one or more from the group comprising bacterial toxins, disseminated intravascular coagulopathy, necrotizing fasciitis, haemorrhagic shock following viral infection, in particular caused by filovirus, arenaviridae, bunyaviridae, flavivirus, dengue, acute hemorrhagic respiratory failure caused by infectious agents or autoimmune diseases, organ failure after organ injury, in particular myocardial infarction, vascular surgery, clamping of organs, haemorrhagic shock, lung infarction, liver infarction, gut infarction, surgical procedures and stroke, and organ dysfunction of grafted organs.

12. The use according to claim 4, wherein shock is associated with one or more from the group comprising bacterial toxins, disseminated intravascular coagulopathy, necrotizing fasciitis, haemorrhagic shock following viral infection, in particular caused by filovirus, arenaviridae, bunyaviridae, flavivirus, dengue, acute hemorrhagic respiratory failure caused by infectious agents or autoimmune diseases, organ failure after organ injury, in particular myocardial infarction, vascular surgery, clamping of organs, haemorrhagic shock, lung infarction, liver infarction, gut infarction, surgical procedures and stroke, and organ dysfunction of grafted organs.

13. The use according to claim 5, wherein shock is associated with one or more from the group comprising bacterial toxins, disseminated intravascular coagulopathy, necrotizing fasciitis, haemorrhagic shock following viral infection, in particular caused by filovirus, arenaviridae, bunyaviridae, flavivirus, dengue, acute hemorrhagic respiratory failure caused by infectious agents or autoimmune diseases, organ failure after organ injury, in particular myocardial infarction, vascular surgery, clamping of organs, haemorrhagic shock, lung infarction, liver infarction, gut infarction, surgical procedures and stroke, and organ dysfunction of grafted organs.

14. The use according to claim 6, wherein shock is associated with one or more from the group comprising bacterial toxins, disseminated intravascular coagulopathy, necrotizing fasciitis, haemorrhagic shock following viral infection, in particular caused by filovirus, arenaviridae, bunyaviridae, flavivirus, dengue, acute hemorrhagic respiratory failure caused by infectious agents or autoimmune diseases, organ failure after organ injury, in particular myocardial infarction, vascular surgery, clamping of organs, haemorrhagic shock, lung infarction, liver infarction, gut infarction, surgical procedures and stroke, and organ dysfunction of grafted organs.

15. The use according to claim 7, wherein shock is associated with one or more from the group comprising bacterial toxins, disseminated intravascular coagulopathy, necrotizing fasciitis, haemorrhagic shock following viral infection, in particular caused by filovirus, arenaviridae, bunyaviridae, flavivirus, dengue, acute hemorrhagic respiratory failure caused by infectious agents or autoimmune diseases, organ failure after organ injury, in particular myocardial infarction, vascular surgery, clamping of organs, haemorrhagic shock, lung infarction, liver infarction, gut infarction, surgical procedures and stroke, and organ dysfunction of grafted organs.

16. The use according to claims 8, wherein shock is associated with one or more from the group comprising bacterial toxins, disseminated intravascular coagulopathy, necrotizing fasciitis, haemorrhagic shock following viral infection, in particular caused by filovirus, arenaviridae, bunyaviridae, flavivirus, dengue, acute hemorrhagic respiratory failure caused by infectious agents or autoimmune diseases, organ failure after organ injury, in particular myocardial infarction, vascular surgery, clamping of organs, haemorrhagic shock, lung infarction, liver infarction, gut infarction, surgical procedures and stroke, and organ dysfunction of grafted organs.
